(19) United States
(12) Patent Application Publication
(10) Pub. No.: US 2025/0220370 A1
(43) Pub. Date: Jul. 3, 2025

(54) SYSTEM AND METHOD FOR PERFORMING HEARING AID FITTINGS

(71) Applicant: Team IP Holdings, LLC, Fort Worth, TX (US)

(72) Inventors: Dan Riordan, Wilmette, IL (US); Keith L. Davis, Salt Lake City, UT (US); Jill Rodriguez, Grapevine, TX (US); Vinh Nguyen, Garland, TX (US)

(21) Appl. No.: 19/084,268

(22) Filed: Mar. 19, 2025

Related U.S. Application Data
(63) Continuation-in-part of application No. 18/367,313, filed on Sep. 12, 2023.
Application No. 18/367,313 is a continuation-in-part of application No. 17/959,452, filed on Oct. 4, 2022.
(60) Provisional application No. 63/573,821, filed on Apr. 3, 2024.
(60) Provisional application No. 63/252,819, filed on Oct. 6, 2021.

(51) Int. Cl. H04R 25/00

(52) U.S. Cl. CPC H04R 25/70

(57) ABSTRACT

Systems for performing hearing aid fittings include a hearing database and a computing device having a processor. The processor is configured to extract a subset of existing most comfortable level (MCL) curves from a set of existing MCL curves stored at a hearing database communicatively coupled to the computing device. The processor is configured to execute a smoothing process on the subset of existing MCL curves to derive a plurality of representative MCL fitting curves. The processor is configured to automatically select a first one of the representative MCL fitting curves as an initial default MCL fitting curve and receive user adjustment feedback to the initial default MCL fitting curve. The processor is configured to automatically select a second one of the representative MCL fitting curves as a selected MCL fitting curve based on the user adjustment feedback and upload the selected MCL fitting curve to a hearing aid.

### CROSS-REFERENCE TO RELATED APPLICATIONS/INCORPORATION BY REFERENCE

The present application claims priority under 35 U.S.C. § 119(e) to provisional application Ser. No. 63/573,821 filed on Apr. 3, 2024, entitled “SYSTEM AND METHOD FOR PERFORMING HEARING AID FITTINGS.” The present application is a continuation-in-part of U.S. patent application Ser. No. 18/367,313, filed on Sep. 12, 2023, and titled “SYSTEM AND METHOD FOR PERFORMING CONSUMER HEARING AID FITTINGS,” which is a continuation-in-part of U.S. patent application Ser. No. 17/959,452, filed on Oct. 4, 2022, and titled “SYSTEM AND METHOD FOR PERFORMING CONSUMER HEARING AID FITTINGS,” which claims priority under 35 U.S.C. § 119(e) to provisional application Ser. No. 63/252,819 filed on Oct. 6, 2021, entitled “SYSTEM AND METHOD FOR PERFORMING CONSUMER HEARING AID FITTINGS.” Each of the above-referenced applications is hereby incorporated herein by reference in its entirety.

Belitz, et al., “A Machine Learning Based Clustering Protocol for Determining Hearing Aid Initial Configurations from Pure-Tone Audiograms. INTERSPEECH 2019, 2325-2329, 2019, which is incorporated by reference herein in its entirety.

### FIELD

The present disclosure relates to hearing aids. More specifically, the present disclosure relates to systems and methods for performing hearing aid fittings.

### BACKGROUND

Hearing aids (HA) are typically customized for specific users by manufacturers and hearing care professionals (HCP). These customizations improve comfort and acoustic performance particular to a user's unique hearing impairment. The customizations include physical modifications to the device and configuration of electro-acoustic characteristics.

Personal sound amplification products (PSAP) are typically distributed directly to a consumer, without assistance of a hearing care professional. Customizations made available to the user are typically limited to basic adjustments, such as volume control, low resolution equalization, and program selection among pre-programmed generic fittings.

The distinction between hearing aids and personal sound amplification products is disappearing with new regulations, new modes of distribution, and new technological capabilities that bridge the gap between these former U.S. Food and Drug Administration (FDA) designations. For purposes of the present disclosure, personal sound amplification products are considered to be in the same class as hearing aids.

Remote control devices and smart-phone applications are currently available, which allow a user to make basic adjustments to the hearing aid device configuration, such as volume control, program selection, or basic equalization. Some applications also provide for remote communication between the user and a hearing care professional, where the hearing care professional can prepare and send a digital package of fitting information to the user's mobile device, which the user can then load into the hearing aid to change its electro-acoustic performance.

The traditional method of tuning hearing aid parameters to hearing loss of an individual uses a measurement of the individual's ability to detect tones at their hearing threshold (i.e., an audiogram). These measurements are traditionally made by an audiologist in a clinical setting. If a customer later finds they are not satisfied with their hearing aid parameters, their only recourse is to return to the audiologist's office for retuning.

Further limitations and disadvantages of conventional and traditional approaches will become apparent to one of skill in the art, through comparison of such systems with some aspects of the present disclosure as set forth in the remainder of the present application.

### SUMMARY

Certain embodiments of the present technology provide a system and method for performing hearing aid fittings, substantially as shown in and/or described in connection with at least one of the figures.

These and other advantages, aspects and novel features of the present disclosure, as well as details of an illustrated embodiment thereof, will be more fully understood from the following description and drawings.

### BRIEF DESCRIPTION OF THE DRAWINGS

FIG. 1 illustrates a block diagram of an exemplary system configured to provide hearing aid fittings, in accordance with embodiments of the present technology.

FIG. 2 illustrates an exemplary structure of a hearing database, in accordance with embodiments of the present technology.

FIG. 3 illustrates an exemplary graph of mean most comfortable level (MCL) curves averaged over a ten (10) year span, in accordance with embodiments of the present technology.

FIG. 4 illustrates an exemplary graph of mean most comfortable level (MCL) curves for age 67, where all frequencies other than 500 Hz were averaged for each unique value at 500 Hz, and normalized such that the mean of each curve is zero (0), in accordance with embodiments of the present technology.

FIG. 5 is an exemplary flowchart for performing a directed loudness balance test to fine tune an initial most comfortable level (MCL) curve, in accordance with embodiments of the present technology.

FIG. 6 illustrates an exemplary graph of an initial most comfortable level (MCL) curve having user interface elements for providing user adjustments, in accordance with embodiments of the present technology.

FIG. 7A is a flowchart of the operation of exemplary user software for providing hearing aid fittings, in accordance with embodiments of the present technology.

FIG. 7B is a continuation of the user software flowchart of FIG. 7A, illustrating exemplary steps for entering user settings, in accordance with embodiments of the present technology.

FIG. 7C is a continuation of the user software flowchart of FIG. 7A, illustrating exemplary steps for deriving a most comfortable level (MCL) curve from an otoacoustic emissions (OAE) measurement of an ear of a user, in accordance with embodiments of the present technology.

FIG. 7D is a continuation of the user software flowchart of FIG. 7A, illustrating exemplary steps for adjusting an initial most comfortable level (MCL) curve, in accordance with embodiments of the present technology.

### DETAILED DESCRIPTION

Embodiments of the present technology provide a system and method for performing hearing aid fittings. Aspects of the present disclosure provide the technical effect of decreasing an amount of time needed to perform an initial hearing aid fitting as compared to a fitting derived from an audiogram. Various embodiments provide the technical effect of allowing a user to select a fitting curve corresponding to hearing aid parameters tuned according to user preferences. Certain embodiments provide the technical effect of generating an initial, default fitting curve based on an age of a user by analyzing a growing database of hearing aid fittings, where the hearing aid database indicates common hearing aid fitting settings versus frequency (i.e., fitting curves). Aspects of the present disclosure provide the technical effect of allowing selection, by the user in their own environment, of a different fitting curve corresponding to an adjustment of an overall level of the initial, default fitting curve. Once a fitting curve corresponding this overall level is selected, various embodiments provide the technical effect of allowing a user to select another different fitting curve corresponding with spectral balance adjustments (e.g., low frequency versus high frequency) of the fitting curve. The result is a fitting curve that is a close match to the fitting curve obtained via an audiogram.

Various embodiments provide an extensive database of hearing aid fittings to derive initial, default fitting curves and common variation fitting curves. A user interface of a computing device, such as a smart phone, PC, tablet, or the like, is linked to the hearing aid. User interface elements, such as sliders or the like, are provided to adjust the overall level and spectral balance of sounds a user hears until the user is satisfied, the adjustments to the overall level and spectral balance of the initial default fitting curve corresponding with selection of one of the common variation fitting curves. In certain embodiments, a user can also be guided to a desired fitting curve by performing loudness balance tasks between low frequency test sounds and a mid-frequency anchor sound, and between high frequency test sounds and a mid-frequency anchor sound.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Furthermore, the term processor or processing unit, as used herein, refers to any type of processing unit that can carry out the required calculations needed for the various embodiments, such as single or multi-core: Central Processing Unit (CPU), Accelerated Processing Unit (APU), Graphic Processing Unit (GPU), Digital Signal Processor (DSP), Field Programmable Gate Array (FPGA), Application-Specific Integrated Circuit (ASIC), or a combination thereof.

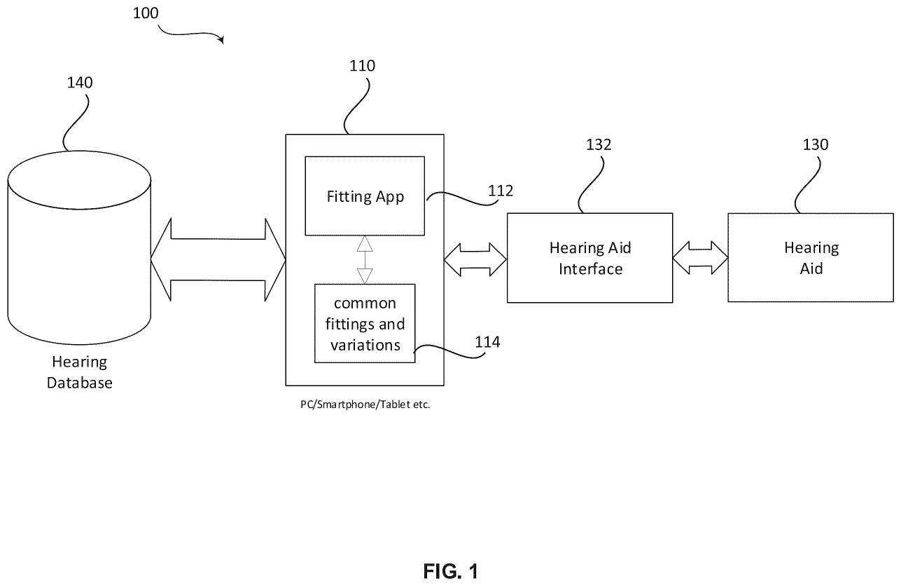

FIG. 1 illustrates a block diagram of an exemplary system 100 configured to provide hearing aid fittings, in accordance with embodiments of the present technology. Referring to FIG. 1, the system 100 includes a computing device 110, a hearing aid 130, and a hearing database 140. The hearing database 140 may exist centrally on another computer system or server, cloud-based, or the like. Additionally and/or alternatively, the hearing database 140 may exist locally (e.g., on the computing device 110). The hearing database 140 comprises hearing related parameters and is a common repository for user hearing related data. The hearing database 140 may be accessible to all of the hearing measurement and related systems.

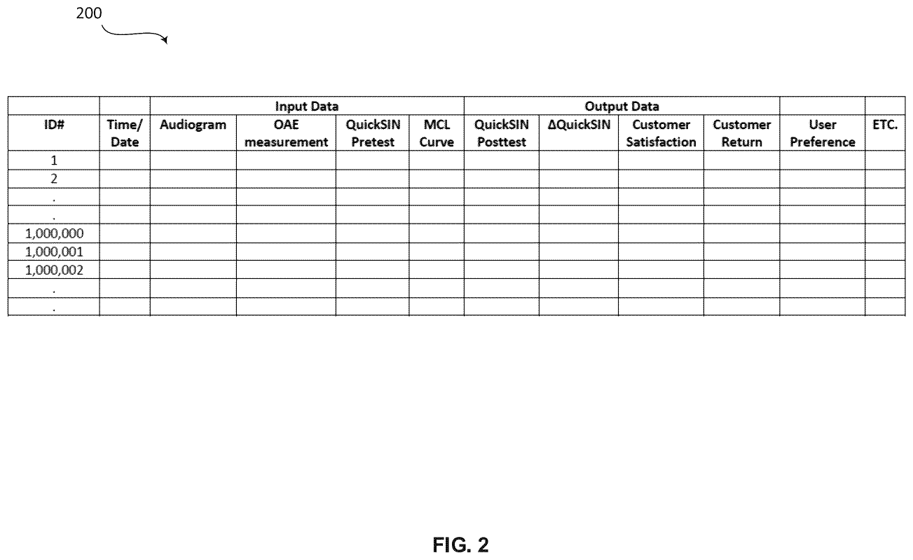

FIG. 2 illustrates an exemplary structure of a hearing database 200, in accordance with embodiments of the present technology. The structure of the hearing database 200 may share various characteristics with the hearing database 140 of FIG. 1. Referring to FIG. 2, each row represents an individual user with possible repeat tests. The columns represent the hearing test data for each user along with their hearing aid fitting, as well as output measures of hearing aid performance as quick speech-in-noise (QuickSIN) improvement, user satisfaction, customer returns, and the like. The characteristic of the database 200 may be of a common form (e.g., structured query language (SQL)) that allows many types of hearing systems to upload and query hearing related data. The first column (ID #) represents a unique user for each entry and each row is a hearing related data entry for a particular user. In various embodiments, the database 200 is anonymous to support privacy requirements. Specifically, while the database 120 may include birthdate, age, and/or other personal information, no real user can be identified by analysis of the data to ensure compliance with the Health Insurance Portability and Accountability Act (HIPAA). Users can have multiple entries such as for different dates when measurement data was collected. Columns 2 to 5 are hearing measurements that have been made. The subsequent columns are outcome measures that relate to how well a set of hearing aid fitting parameters (i.e., most comfortable level (MCL) curves) are valued by the user, such as satisfaction ratings, user preferences, customer return data, and the like.

Referring again to FIG. 1, the hearing aid 130 comprises one or more microphones, one or more receivers, memory, one or more processors, and communication connections. The one or more microphones are configured to receive sound exterior to an ear canal. The microphones convert the sound to electrical signals and provide the electrical signals to the one or more processors. The one or more processors modify the sound level by applying hearing aid parameters retrieved from memory and/or received from the computing device 110. The one or more processors pass the electrical signals having the modified sound level to the receiver. The receiver converts the electrical signals to sound, which is communicated from the receiver to a user's ear canal. The memory, one or more processors, and communication connections of the hearing aid 130 may share various characteristics with the memory, one or more processors, and communication connections as described below with respect to the computing device 110. The hearing aid 130 comprises a hearing aid interface 132 that comprises suitable logic, circuitry, interfaces, and/or code that is operable to transmit and receive information with the computing device 110. The hearing aid interface 132 may comprise a hearing aid docking station, a wired interface, and/or a wireless interface (e.g., transceiver), for example.

The computing device 110 may comprise, for example, a smart phone, a tablet computer, a personal computer, or any suitable electronic device capable of communication with the hearing aid 130 and hearing database 140 via wired or wireless connections, such as Bluetooth, BLE, short-range, long range, Wi-Fi, cellular, personal communication system (PCS), USB, or any suitable wired or wireless connection. The computing device 110 may be realized in a centralized fashion in at least one computer system, or in a distributed fashion where different functionality of the computing device 110 is spread across several interconnected computer systems.

The computing device 110 may include a display, user input devices, a memory, one or more processors, one or more communication connections, and the like. The display may be any device capable of communicating visual information to a user. For example, a display may include a liquid crystal display, a light emitting diode display, and/or any suitable display. The display can be operable to display information from a software application, such as a hearing aid fitting application 112, or any suitable information. In various embodiments, the display may display information provided by the one or more processors, for example.

The user input device(s) may include a touchscreen, button(s), motion tracking, orientation detection, voice recognition, a mousing device, keyboard, camera, and/or any other device capable of receiving a user directive. In certain embodiments, one or more of the user input devices may be integrated into other components, such as the display, for example. As an example, the user input device may include a touchscreen display.

The memory may be one or more computer-readable memories, such as compact storage, flash memory, random access memory, read-only memory, electrically erasable and programmable read-only memory and/or any suitable memory, for example. The memory may include databases, libraries, sets of information, or other storage accessed by and/or incorporated with the one or more processors, for example. The memory may be able to store data temporarily or permanently, for example. The memory may be capable of storing data generated by the one or more processors and/or instructions readable by the one or more processors, among other things. In various embodiments, the memory stores information related to a hearing aid fitting application 112 and a common fittings and variations database 114, for example.

The communication connection(s) allow communication between the computing device and other external systems, such as the hearing aid 130 and the hearing database 140, for example. The communication connection(s) may include wired and/or wireless interfaces. The wireless interfaces may include transceivers, such as Bluetooth, short-range, long range, Wi-Fi, cellular, personal communication system (PCS), or any suitable transceiver.

The one or more processors may be one or more central processing units, microprocessors, microcontrollers, and/or the like. The one or more processors may be an integrated component, or may be distributed across various locations, for example. The one or more processors may be capable of executing a software application (e.g., the hearing aid fitting application 112, among other things), receiving input information from a user input device and/or communication connection(s), and generating an output displayable by a display, among other things. The one or more processors may comprise suitable logic, circuitry, interfaces, or code configured to query and update the hearing database 140, identify initial default hearing aid fitting parameters (i.e., most comfortable level (MCL) curves) based on a user's age or OAE measurement, derive common variations of the initial default fitting curves corresponding with adjustments to the overall level and spectral balance of the initial default fitting curves, and upload the selected fitting curves to the user's hearing aid 130. In certain embodiments, the one or more processors may store the derived common variations of the initial default fitting curves at the common fittings and variations database 114 and/or the hearing database 140. In various embodiments, the one or more processors may communicate via communication connection(s) with the hearing database 140 to perform hearing aid parameter queries and store adjusted hearing aid parameters, for example. In an exemplary embodiment, the one or more processors may communicate via communication connection(s) with the hearing aid 130 to upload the hearing aid fitting parameters. For example, the one or more processors may send the initial default fitting curve and the selected common variation of the fitting curve after user adjustment to the hearing aid devices 130.

The one or more processors may comprise suitable circuitry, logic, interfaces, and/or code configured to derive hearing aid fitting parameters (i.e., MCL curves) from previously used fitting parameters extracted from the hearing database 140, 200. For example, only the most recent hearing device fitting parameters of patients that retained their hearing aids for at least one year may be extracted. Alternatively, the average hearing device fitting parameters per patient may be extracted. The extracted set of desirable hearing device fitting parameters may be processed using a variety of quantitative methods, such as the averaging method, principal component analysis (PCA) method, and/or neural network method discussed below, to generate a set of optimal representative curves that can be discretely traversed and selected by a user through a user interface. For example, the MCL curves may correspond with an initial default fitting curve and common variations of the fitting curve selectable based on user adjustments. In various embodiments, the set of optimal representative curves may be stored at the common fittings and variations database 114 and/or the hearing database 140.

### The Averaging Method

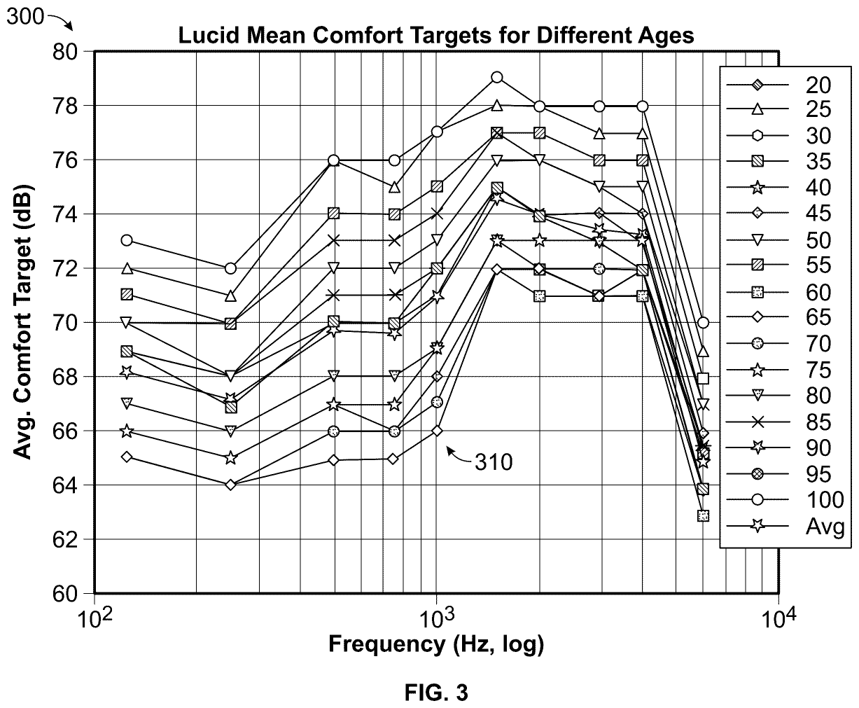

In various embodiments, representative fitting curves are created by averaging all values per frequency for a particular age. The mean MCL spectral shape appears relatively constant with age with mean variations at any age being shifted versions of the overall mean. FIG. 3 illustrates an exemplary graph 300 of mean most comfortable level (MCL) curves 310 averaged over a ten (10) year span, in accordance with embodiments of the present technology.

Individual variation of curve shape across MCLs, however, can be large. Consequently, fitting curves can be created by averaging values only at frequencies except those with the most variation. The below table of statistics shows that for a sampling of all customer fitting curves, 500 Hz shows the highest standard deviation out of eight possible frequencies.

| Statistic | Age | Hz250 | Hz500 | Hz750 | Hz1000 | Hz1500 | Hz2000 | Hz3000 | Hz4000 |
|---|---|---|---|---|---|---|---|---|---|
| mean | 74.3 | 66.6 | 68.9 | 68.8 | 70.3 | 74.5 | 74.2 | 73.9 | 73.9 |
| std | 11.4 | 10.4 | 12.2 | 11.6 | 11.3 | 9.6 | 9.7 | 10.3 | 10.7 |
| min | 20 | 37 | 37 | 37 | 37 | 39 | 36 | 36 | 36 |
| 25% | 68 | 60 | 61 | 61 | 62 | 68 | 68 | 67 | 67 |
| 50% | 75 | 66 | 68 | 68 | 70 | 74 | 74 | 74 | 74 |
| 75% | 82 | 73 | 77 | 76 | 78 | 81 | 80 | 81 | 81 |
| max | 100 | 122 | 122 | 127 | 132 | 127 | 127 | 122 | 117 |

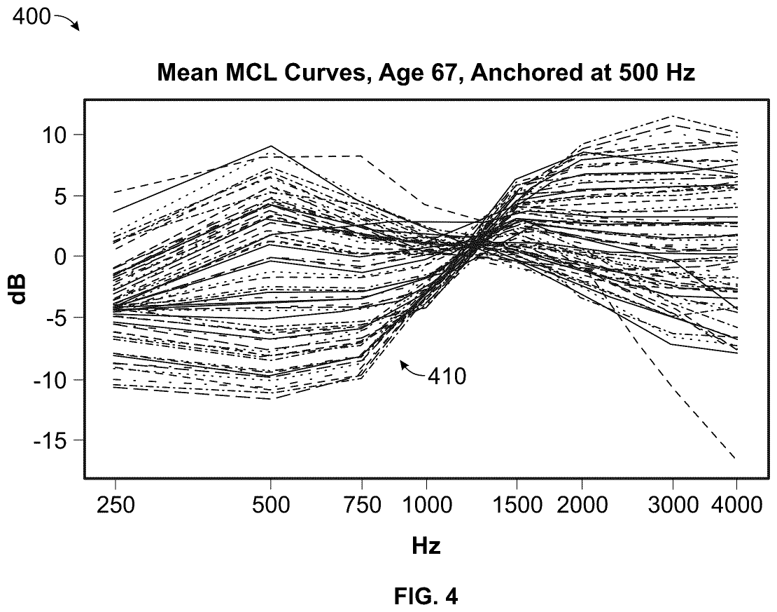

FIG. 4 illustrates an exemplary graph 400 of mean most comfortable level (MCL) curves 410 for age 67, where all frequencies other than 500 Hz were averaged for each unique value at 500 Hz, and normalized such that the mean of each curve is zero (0), in accordance with embodiments of the present technology. Referring to FIG. 4, for each unique value at 500 Hz, the average values for each of the other frequencies (250 Hz, 750 Hz, 1000 Hz, 1500 Hz, 2000 Hz, 3000 Hz, 4000 Hz) were computed. The curves are then considered to be anchored around 500 Hz. To account for different overall volume levels, the cross-frequency mean for each curve was subtracted, resulting in curves that are anchored around 500 Hz whose mean is zero.

### The Principal Component Analysis (PCA) Method

Representative fitting curves can also be derived by smoothing raw fitting curves through a dimensionality reduction method, such as principal component analysis, where raw fitting curves in f dimensions (such as f number of frequencies recorded) are reduced into c dimensions such that c<f, and subsequently uncompressed back into f dimensions with some loss of information. The process starts with computing the f×f covariance matrix C between each dimension d:

$$C = \begin{matrix}Cov ( {d}_{1} , {d}_{1} ) & … & Cov ( {d}_{1} , {d}_{f} ) \\ ⋮ & ⋱ & ⋮ \\ Cov ( {d}_{f} , {d}_{1} ) & … & Cov ( {d}_{f} , {d}_{f} )\end{matrix}$$

The eigenvalues λ and matrix of eigenvectors X of the covariance matrix are then computed such that CX=λX:

$$X = \begin{matrix}{w}_{1 , 1} & … & {w}_{f , 1} \\ ⋮ & ⋱ & ⋮ \\ {w}_{1 , f} & … & {w}_{f , f}\end{matrix} \\ λ = \begin{matrix}λ1 & … & λ f\end{matrix}$$

The eigenvalues are then sorted from largest to smallest. The eigenvector corresponding to the largest eigenvalue is the feature vector of the first principal component. Similarly, the eigenvector corresponding to the second largest eigenvalue is the second principal component. The first principal component would account for the largest amount of variance in the data, and the second principal component would account for the second largest amount of variance in the data, and so on. The amount of variance explained by eigenvector w1 is equal to

$$\frac{λ1}{Σλ} .$$

The original data can be reduced by D_{reduced}=D_{original}×X_{c}, where X_{c} contains the first c column vectors of X. D_{transformed} is subsequently inverse transformed into f original frequencies using the full eigenvector matrix X by Dₛₘₒₒₜₕ=D_{reduced}×X. The result is a family of representative fitting curves that retain only variations that can be explained using c×f linear coefficients.

### The Neural Network Method

Another method of deriving optimal representative fitting curves is to use an encoder-decoder neural network architecture on a sample dataset at a given age. The neural network attempts to encode and then decode the curves, while learning optimal encoding parameters and decoding parameters in the process. The result is a family of optimal representative fitting curves that retain only systemic variations in the original curves.

The encoder network consists of the input layer of f dimensions, at least one hidden layer H of n₁ nodes, and an output layer of c dimensions such that c<f. The decoder network consists of the encoder's output layer as the input, at least one hidden layer of n nodes, and an output layer of f nodes. n1 sets of f weights, w₀ . . . w_{f} are initialized at random or at zero, with w₀ as the bias. Each entry d of f dimensions in the input data is multiplied with each of the n₁ sets of weights, the results of which are passed through an activation function F, such as the sigmoid function

$$\frac{1}{1 + {e}^{- x}} ,$$

to determine whether each of the n₁ nodes is activated:

$$Activation = F ( x ) = \frac{1}{1 + {e}^{- x}} , where x = {w}_{0} + {\sum }_{i = 1}^{f} {d}_{i} {w}_{i}$$

Each node in the hidden layer H is assigned a value of 0 (deactivated) or 1 (activated). For each dimension in the output layer, the hidden nodes are subsequently multiplied with c sets of n₁ weights w₀ . . . wₙ₂, also initialized at random or at zero, with w, as the bias, but without being fed through an activation function:

$$Encoded output = {w}_{0} + {\sum }_{i = 1}^{f} {H}_{i} {w}_{i}$$

The encoded output of c dimensions is then fed through the same activation process as the initial input, but for n₂ nodes instead of n₁. The activated/deactivated n₂ nodes are then multiplied by f sets of n₂ weights w₀ . . . wₙ₂ to provide the decoded output of f dimensions. The decoded output is then compared with the original input using a loss function, such as mean squared error (MSE). The derivatives of the loss function with respect to each set of weights are computed, and the weights are adjusted in the direction that minimizes loss, known as gradient descent. The adjusted weights are used on the next batch of input data to iteratively minimize the difference between the decoded output and the original input while compressing f dimensions to c dimensions in the encoding step, resulting in smoothed fitting curves.

Regardless of the method of derivation, the set of unique optimal representative fitting curves ultimately includes curves with a higher emphasis in the low frequencies and curves with a higher emphasis on higher frequencies, given the same overall volume. Furthermore, additional optimal fitting curves may be generated between the curves already observed. In all cases described, the final set of potential hearing device fitting curves are discretely traversed by the user, who ultimately selects the desired fitting curve through a user interface (e.g., by providing adjustments to the initial default fitting curve, the adjustments corresponding with different fitting curves in the set of potential hearing device fitting curves).

Note that in all of these techniques, the algorithm and predictions are dynamic as the data set increases with time, correspondingly increasing optimum hearing aid identification performance with time.

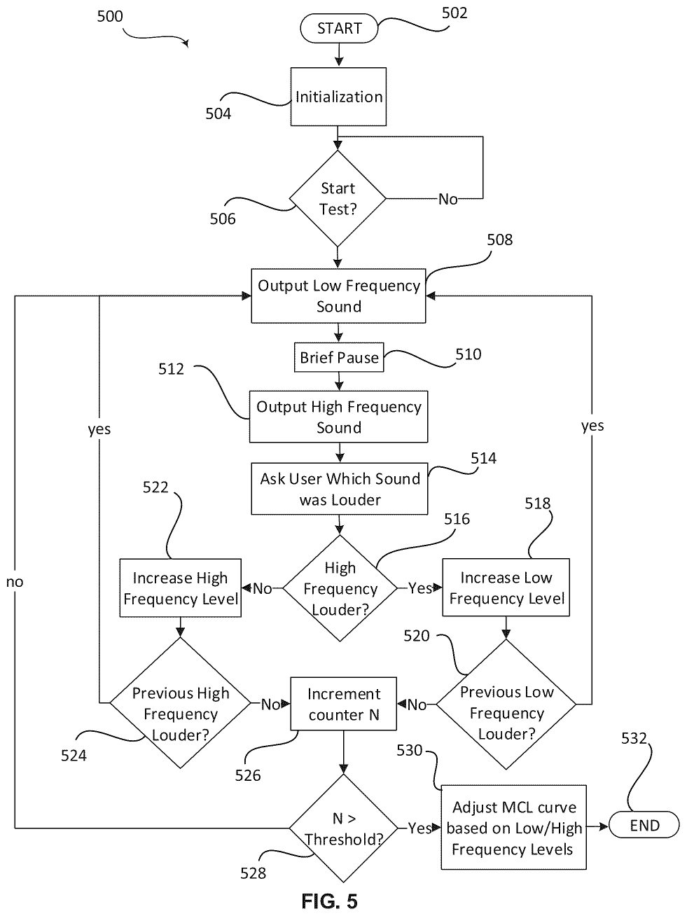

FIG. 5 is an exemplary flowchart 500 for performing a directed loudness balance test to fine tune an initial most comfortable level (MCL) curve, in accordance with embodiments of the present technology. Referring to FIG. 5, there is shown a flow chart 500 comprising exemplary steps 502 through 532. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

The method begins at step 502 and includes initialization at step 504, such as retrieving an initial MCL curve, for example, based on the user's age. The starting sound levels of the loudness balance test are set to the respective low and high frequency levels of the starting MCL curve, and a loudness balance counter is reset to zero (0). The at least one processor of the computing device 110 receives an input from a user at step 506 to start the loudness balance test and proceeds to step 508 where the computing device 110 directs the hearing aid 130 to output a low frequency sound for approximately one second, or any suitable duration. After a brief pause at step 510, the computing device 110 directs the hearing aid 130 to output a high frequency sound for approximately one second, or any suitable duration, at step 512. A user of the hearing aid 130 compares the loudness of different sounds to establish the desired spectral tilt. The test sounds can be one or more tones, band-limited noise or speech, or any suitable test sounds. The frequency range for the low frequency sound may be in the range of 250 to 750 Hz, or any suitable low frequency range. The frequency range of the high frequency sound may be in the range of 2000 to 6000 Hz, or any suitable high frequency range.

At step 514, the at least one processor of the computing device 110 outputs a question via a user interface, asking the user which sound was louder. At step 516, a user provides a response. If the user indicates the high frequency sound was louder, then the method 500 proceeds to step 518 where an MCL curve having an increased low frequency level is selected. In subsequent iterations of outputting the test tones, a loudness balance counter is incremented at step 526 if the computing device determines at step 520 that the user had indicated that the low frequency level was louder for the last outputted test tones. If the computing device determines at step 520 that the user had indicated that the high frequency level was louder for the last outputted test tones, the method returns to step 508 without incrementing the counter.

If the user indicates at step 516 that the low frequency sound was louder, then the method 500 proceeds to step 522 where an MCL curve having an increased high frequency level is selected. A loudness balance counter is incremented at step 526 if the computing device determines at step 524 that the user had indicated that the high frequency level was louder for the last outputted test tones. If the computing device determines at step 524 that the user had indicated that the low frequency level was louder for the last outputted test tones, the method returns to step 508 without incrementing the counter. Basically, the counter is incremented at step 526 every time a response switches from low to high or high to low in subsequent trials. After incrementing the counter at step 526, if the number N of the counter does not exceed a threshold at step 528, the process 500 returns to step 508 until the at least one processor of the computing device 110 determines at step 528 that the number N of the counter exceeds the threshold. The loudness balance test is finished after a sufficient number of switches occur, for example, three. At step 530, the MCL curve is selected to match the levels of the low and high frequencies that produced a perceived equal loudness pursuant to the user adjustments during the test.

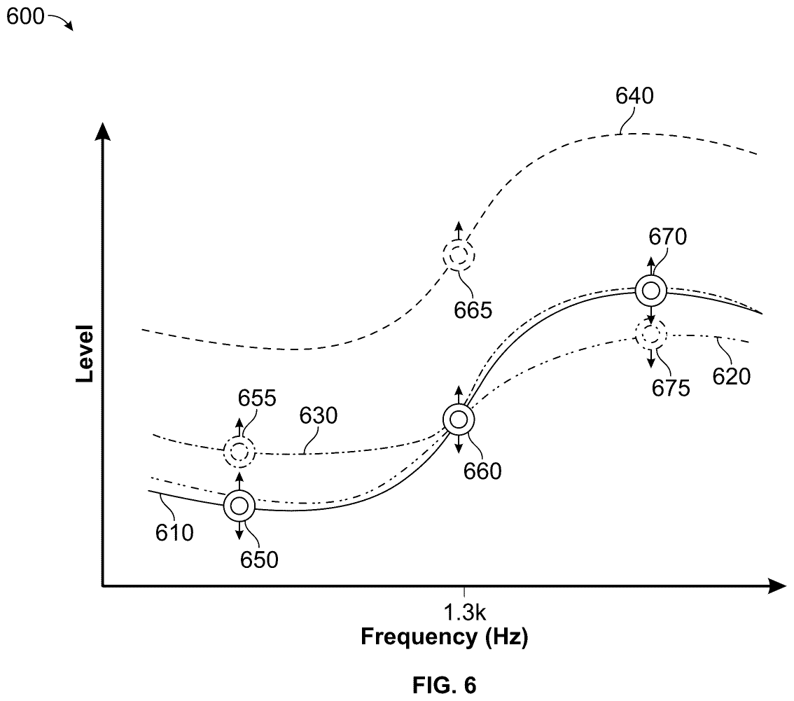

FIG. 6 illustrates an exemplary graph 600 of an initial most comfortable level (MCL) curve 610 having user interface elements 650-675 for providing user adjustments, in accordance with embodiments of the present technology. Referring to FIG. 6, an initial default MCL curve 610 is shown. A second MCL curve 620 may be selected based on a reduction at high frequencies. A third MCL curve 630 may be selected based on an increase at low frequencies. A fourth MCL curve 640 may be selected based on an increase at mid frequencies. A user interface element 650, 655, such as a handle or slider, is shown at the low frequencies at an initial position 650 and a higher position 655. A user interface element 660, 665, such as a handle or slider, is shown at the mid frequencies at an initial position 660 and a higher position 665. A user interface element 670, 675, such as a handle or slider, is shown at the high frequencies at an initial position 670 and a lower position 675. In an exemplary embodiment, a low frequency adjustment may result in an MCL fitting curve being selected that has a different (i.e., higher or lower) low frequency while substantially maintaining the mid and high frequency levels. For example, if the low frequency is adjusted higher using a user interface element 650, 655 or in response to the loudness balance test as described above with respect to FIG. 5, the low frequencies may be increased while substantially maintaining the mid and high frequencies as shown in FIG. 6. A high frequency adjustment may result in an MCL fitting curve being selected that has a different (i.e., higher or lower) high frequency while substantially maintaining the low and mid frequency levels. For example, if the high frequency is adjusted lower using a user interface element 670, 675 or in response to the loudness balance test as described above with respect to FIG. 5, the high frequencies may be decreased while substantially maintaining the low and mid frequencies as shown by the bottom curve 620 in FIG. 6. A mid frequency adjustment may result in an MCL fitting curve being selected with all frequencies being adjusted higher or lower. For example, if the mid frequency is adjusted higher using a user interface element 660, 665, all frequencies may be increased as shown in FIG. 6.

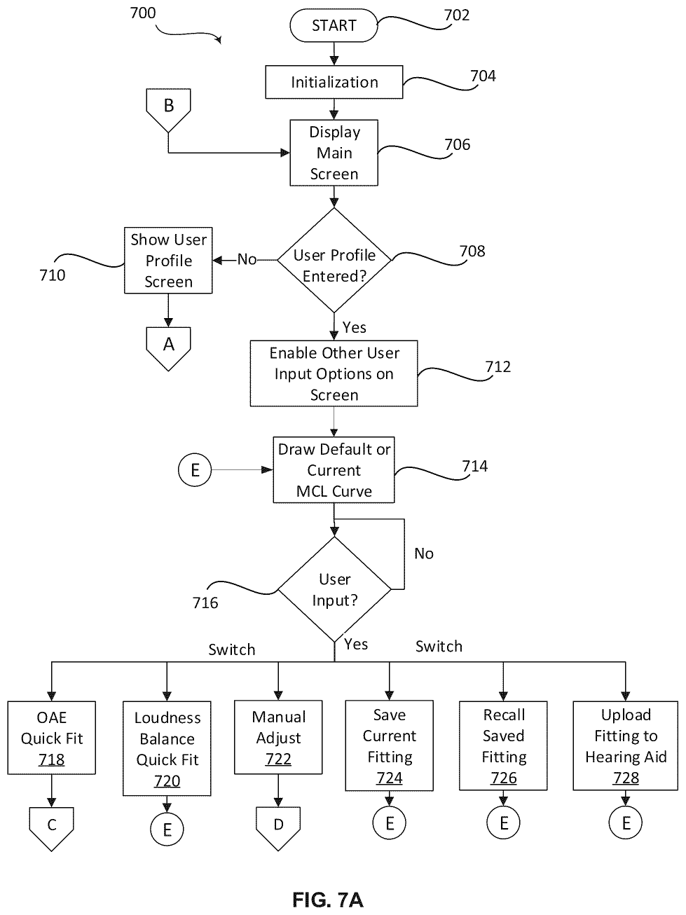

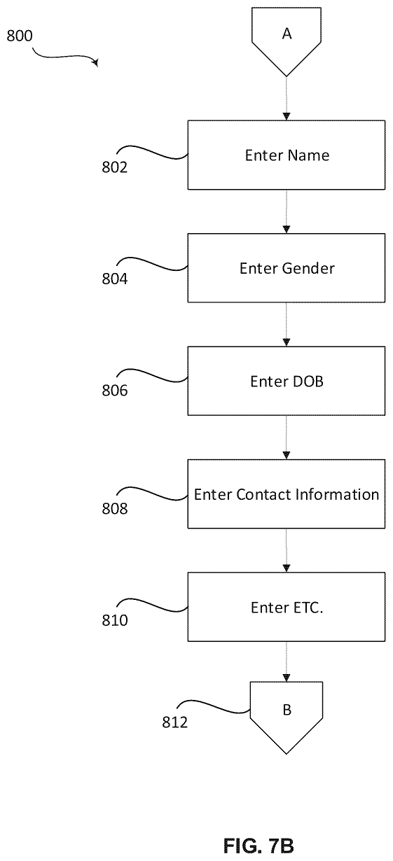

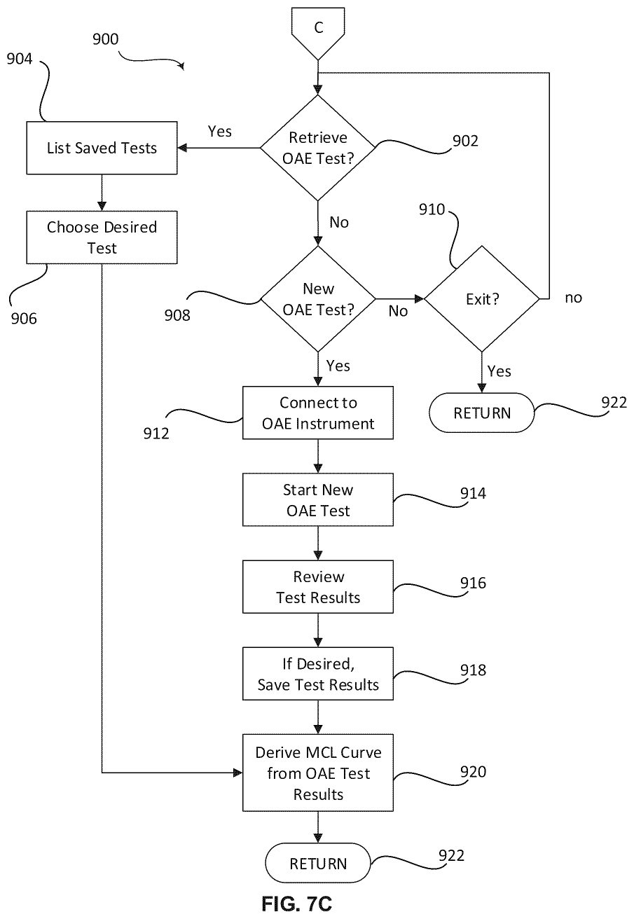

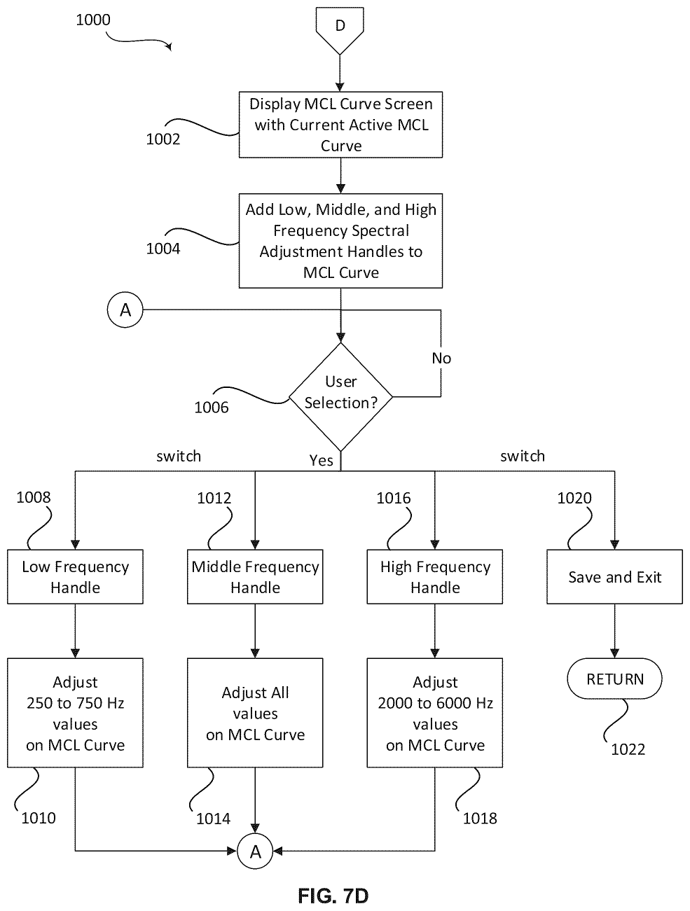

FIG. 7A is a flowchart of the operation of exemplary user software for providing hearing aid fittings, in accordance with embodiments of the present technology. Referring to FIG. 7A, there is shown a flow chart 700 comprising exemplary steps 702 through 728. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

The method begins at step 702 and includes initialization of the software at step 704. At step 706 a main screen is presented at a display of the computing system 110. In various embodiments, the main screen may include user selectable options, such as a user profile option, an otoacoustic emissions (OAE) quick fit option, a loudness balance quick fit option, a manual adjust option, a save current fitting option, a recall saved fitting option, and an upload fitting to hearing aid option, among other things. If a user profile has not been previously input, the other options may be grayed out and/or otherwise unavailable until the user profile has been entered. The at least one processor of the computing system 110 executing the software determines whether a user profile has been entered at step 708. If not, the process 700 continues to step 710 where a user profile screen is shown, and the process 800 of FIG. 7B is performed as described below. Once the at least one processor of the computing system 110 executing the software determines that the user profile has been entered at step 708, the other selectable user input options may be enabled on the main screen at step 712. At step 714, an MCL curve is selected for presentation at the display of the computing system 110. At the start, the MCL curve may be an initial derived MCL curve selected based on the user profile information, such as the user age. Subsequently, the MCL curve may be selected based on user adjustment feedback provided at steps 718, 720, or 722.

At step 716, the at least one processor of the computing system 110 executing the software application awaits a user input of a selectable option, such as the otoacoustic emissions (OAE) quick fit option, the loudness balance quick fit option, the manual adjust option, the save current fitting option, the recall saved fitting option, or the upload fitting to hearing aid option. If the computing system 110 receives the OAE quick fit option, the process 700 continues to step 718 where the method 900 of FIG. 7C is performed and returns to step 714 to present a current MCL curve selected based on the OAE measurement. If the computing system 110 receives the loudness balance option, the process 700 continues to step 720 where the method 500 of FIG. 5 is performed and returns to step 714 to present a current MCL curve selected based on the loudness balance test. If the computing system 110 receives the manual adjust option, the process 700 continues to step 722 where the method 1000 of FIG. 7D is performed and returns to step 714 to present a current MCL curve selected based on the manual frequency adjustments. If the computing system 110 receives the save current fitting option, the process 700 continues to step 724 where the current MCL curve presented at step 724 is stored at memory of the computing system 110 and/or at the hearing database 140, 200, and the process 700 returns to step 714. If the computing system 110 receives the recall saved fitting option, the process 700 continues to step 726 where a saved fitting may be selected and presented at the display of the computing system 110 at step 714. If the computing system 110 receives the upload fitting to hearing aid option, the process 700 continues to step 728 where the computing system uploads the current fitting to the hearing aid 130 via the hearing aid interface 132, and the process 700 returns to step 714.

FIG. 7B is a continuation of the user software flowchart 700 of FIG. 7A, illustrating exemplary steps 800 for entering user settings, in accordance with embodiments of the present technology. Referring to FIG. 7B, there is shown a flow chart 800 comprising exemplary steps 802 through 812. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below. Still referring to FIG. 7B, the user may enter or review settings that identify the user, such as name at step 802, gender at step 804, date of birth at step 806, contact information at step 808, and the like at step 810. The settings data may be used to retrieve and/or store hearing aid fitting parameters from/to the hearing database 140, 200 as illustrated in FIGS. 1 and 2. At step 812 the process may return to displaying the main screen at step 706 of FIG. 7A.

FIG. 7C is a continuation of the user software flowchart 700 of FIG. 7A, illustrating exemplary steps 900 for deriving a most comfortable level (MCL) curve from an otoacoustic emissions (OAE) measurement of an ear of a user, in accordance with embodiments of the present technology. Referring to FIG. 7C, there is shown a flow chart 900 comprising exemplary steps 902 through 922. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 902, at least one processor of a computing system 110 executing the software application may receive an input to retrieve an OAE test at step 902 and/or start a new OAE test at step 908. If the user does not desire to retrieve an OAE test or perform a new test, the user may exit at step 910 and return to step 714 of FIG. 7A at step 922. If the computing system 110 receives a user input to retrieve an OAE test at step 902, a list of saved tests may be presented at the display of the computing system 110 at step 904. At step 906, the computing system 110 receives a selection of one of the saved OAE tests and an MCL curve is derived from the OAE test results at step 920 as explained below. If the computing system 110 receives a user input to perform a new OAE test at step 904, the computer system 110 may connect to an OAE instrument at step 912 and start a new OAE test at step 914. The OAE test results may be presented at a display of the computing system 110 for review at step 916 and the OAE test results may be stored at step 918. At step 920 an MCL curve may be derived from the selected retrieved OAE test results or the new OAE test results. For example, the hearing database 140 and/or the common fitting and variations database 114 may be searched for an optimal MCL curve based on the OAE measurement. The OAE measurement forms the input into an algorithm configured to identify an optimal MCL curve with a similar OAE input. The algorithm, executed by the computing device 110 or hearing database 140, queries the data in the hearing database 140 and/or the common fitting and variations database 114 using a statistical method to identify the optimal MCL curve. For example, the algorithm may analyze the hearing data to identify a correlation or regression relationship between OAE data and MCL curve for the subset of data from satisfied customers. As another example, the algorithm may analyze the hearing data to identify MCL curves that produced a largest improvement in QuickSIN scores for a given OAE data. As another example, a machine learning algorithm may be trained from a set of OAE measurements, age, and associated fitting parameters from the hearing database 140 to form a model that can predict optimal MCL curves given any OAE measurement.

In various embodiments, machine-learning techniques may be employed to obtain the optimum MCL curves from OAE data at step 920. For example, using supervised machine learning, the output measures of delta QuickSIN scores, customer satisfaction ratings, and/or user preferences could be used with an audiogram and OAE input data to train a neural network to predict the optimal MCL curve. It has also been shown how to use unsupervised learning to find natural clusters of fittings from audiograms. See e.g., Belitz, et al., “A Machine Learning Based Clustering Protocol for Determining Hearing Aid Initial Configurations from Pure-Tone Audiograms. INTERSPEECH 2019, 2325-2329, 2019, which is incorporated by reference herein in its entirety. OAE data could be included in this clustering analysis to improve this technique. Note that in all of these techniques, the algorithm and predictions are dynamic as the data set increases with time, correspondingly increasing optimum hearing aid identification performance with time. Certain embodiments may be used in conjunction with interactive fine-tuning adjustment techniques, where an optimum prediction fitting, based on OAE and other objective measures, may be further personalized using an interactive fine-tuning adjustment system.

At step 922, the process 900 is finished and may return to step 714 of FIG. 7A where the MCL curve corresponding with the OAE measurement is presented at a display of the computing device 110.

FIG. 7D is a continuation of the user software flowchart 700 of FIG. 7A, illustrating exemplary steps 1000 for adjusting an initial most comfortable level (MCL) curve, in accordance with embodiments of the present technology. Referring to FIG. 7D, there is shown a flow chart 1000 comprising exemplary steps 1002 through 1022. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

FIG. 7D corresponds with the Manual Adjust option 722 of FIG. 7A. In an exemplary embodiment, as illustrated in FIG. 6, the current MCL curve may be presented at the display of the computing system 110 with three user interface elements on the curve that a user selects to change the shape of the curve. Selecting and moving the low frequency handle (left) moves the curve values between 250 and 750 Hz. Similarly, the high frequency handle (right) moves the curve values between 2000 and 6000 Hz. The middle frequency handle (center) is located at 1300 Hz and moves the entire curve up and down. The new points accompanying each curve change are selected from the set of MCL curves in the common fittings and variations database 114.

More specifically, at step 1002, at least one processor of the computing system 110 executing the software application presents a current MCL curve. At step 1004, user interface elements such as sliders or handles are provided on the current MCL curve, the user interface elements corresponding with low, middle, and high frequency spectral adjustments. At step 1006, at least one tone corresponding with the current MCL curve is output and a user selection of the low frequency handle, middle frequency handle, high frequency handle, or a save and exit option is received. If the user selection is an adjustment of the low frequency handle (i.e., left user interface element in FIG. 6) at step 1008, a different MCL curve is selected having the adjusted low frequency (e.g., 250 to 750 Hz) at step 1010 and the process returns to output at least one tone corresponding with the different MCL curve and to await a user selection at step 1006. If the user selection is an adjustment of the middle frequency handle (i.e., center user interface element in FIG. 6) at step 1012, a different MCL curve is selected having the all frequencies adjusted at step 1014 and the process returns to output at least one tone corresponding with the different MCL curve and to await a user selection at step 1006. If the user selection is an adjustment of the high frequency handle (i.e., right user interface element in FIG. 6) at step 1016, a different MCL curve is selected having the adjusted high frequency (e.g., 2000 to 6000 Hz) at step 1018 and the process returns to output at least one tone corresponding with the different MCL curve and to await a user selection at step 1006. Once the user is satisfied with the adjustments, the selected MCL curve is saved at step 1020. At step 1022, the process 1000 ends and may return to step 714 of FIG. 7A where the MCL curve selected based on the Manual Adjust option 722, 1000 is presented at a display of the computing device 110.

Aspects of the present disclosure provide a system 100 and method 500, 700, 800, 900, 1000 for performing hearing aid fittings. The system 100 comprises a hearing database 140, 200 and a computing device 110. The hearing database 140, 200 is configured to store hearing data comprising a set of existing most comfortable level (MCL) curves. The computing device 110 may comprise at least one processor. The computing device 110 may be communicatively coupled to the hearing database 140, 200 and a hearing aid 130. The at least one processor may be configured to extract a subset of existing most comfortable level (MCL) curves from the set of existing MCL curves. The subset of existing MCL curves is less than the set of MCL curves. The at least one processor may be configured to execute a smoothing process on the subset of existing MCL curves to derive a plurality of representative most comfortable level (MCL) fitting curves 310, 410, 610, 620, 630, 640. The at least one processor may be configured to automatically select a first one 610 of the plurality of representative MCL fitting curves 310, 410, 610, 620, 630, 640 as an initial default most comfortable level (MCL) fitting curve 610. The at least one processor may be configured to receive user adjustment feedback 500, 718, 720, 722, 900, 1000 to the initial default MCL fitting curve 610. The at least one processor may be configured to automatically select a second one 620, 630, 640 of the plurality of representative MCL fitting curves 310, 410, 610, 620, 630, 640 as a selected most comfortable level (MCL) fitting curve 620, 630, 640 based on the user adjustment feedback 500, 718, 720, 722, 900, 1000. The at least one processor may be configured to upload the selected MCL fitting curve 620, 630, 640 to the hearing aid 130.

In an exemplary embodiment, the one 610 of the plurality of representative MCL fitting curves 310, 410, 610, 620, 630, 640 is automatically selected as the initial default MCL fitting curve 610 based on a user age. In a representative embodiment, the system 100 comprises the hearing aid 130 configured to apply the selected MCL fitting curve 620, 630, 640 to generate an acoustic output. In various embodiments, the at least one processor is configured to execute the smoothing process to average all MCL values in the subset of existing MCL curves corresponding with one user age for each of a predetermined plurality of frequencies, to derive the plurality of representative MCL fitting curves 310, 410, 610, 620, 630, 640. In certain embodiments, the at least one processor configured to execute the smoothing process to average MCL values in the subset of existing MCL curves corresponding with one user age for each of a predetermined plurality of frequencies except for one of the predetermined plurality of frequencies having a highest variation of the MCL values, to derive the plurality of representative MCL fitting curves 310, 410, 610, 620, 630, 640. In an exemplary embodiment, the at least one processor configured to execute the smoothing process to apply principal component analysis to reduce a dimensionality of the subset of existing MCL curves from a first number of frequencies to a second number of frequencies that is less than the first number of frequencies, and uncompress the second number of frequencies back to the first number of frequencies with some loss of information to derive the plurality of representative MCL fitting curves 310, 410, 610, 620, 630, 640. In a representative embodiment, the at least one processor is configured to execute the smoothing process to apply an encoder-decoder neural network architecture to the subset of existing MCL curves to derive the plurality of representative MCL fitting curves 310, 410, 610, 620, 630, 640.

In various embodiments, the user adjustment feedback 500, 718, 720, 722, 900, 1000 is received in response to a loudness balance test 500, 720. The loudness balance test 500, 520 may comprise: (1) separately outputting 508, 512 a low frequency sound and a high frequency sound from the hearing aid 130; (2) receiving a user input 516 indicating a user of the hearing aid 130 perceived one the low frequency sound and the high frequency sound as louder; (3) increasing a level 518, 522 of the one of the low frequency sound or the high frequency sound that was perceived as louder; and (4) repeating (1)-(3) until a defined condition is met 528. In certain embodiments, the user adjustment feedback 500, 718, 720, 722, 900, 1000 is results of an otoacoustic emissions (OAE) test 718, 900. In an exemplary embodiment, the user adjustment feedback 500, 718, 720, 722, 900, 1000 corresponds with received manual adjustments 722, 1000 of one or more user interface elements 650-675 of a plurality of user interface elements 650-675, each of the plurality of user interface elements 650-675 corresponding with a respective frequency range and defining a level of the respective frequency range.

Various embodiments provide a system 100 and method 500, 700, 800, 900, 1000 for performing hearing aid fittings. The method 500, 700, 800, 900, 1000 comprises extracting, by at least one processor of a computing device 110, a subset of existing most comfortable level (MCL) curves from a set of existing most comfortable level (MCL) curves stored at a hearing database 130, 200 communicatively coupled to the computing device 110. The subset of existing MCL curves is less than the set of MCL curves. The method 500, 700, 800, 900, 1000 comprises deriving, by the at least one processor executing a smoothing process on the subset of existing MCL curves, a plurality of representative most comfortable level (MCL) fitting curves 310, 410, 610, 620, 630, 640. The method 500, 700, 800, 900, 1000 comprises automatically selecting 714, by the at least one processor, a first one 610 of the plurality of representative MCL fitting curves 310, 410, 610, 620, 630, 640 as an initial default most comfortable level (MCL) fitting curve 610. The method 500, 700, 800, 900, 1000 comprises receiving 716, by the at least one processor, user adjustment feedback 500, 718, 720, 722, 900, 1000 to the initial default MCL fitting curve 610. The method 500, 700, 800, 900, 1000 comprises automatically selecting 714, by the at least one processor, a second one 620, 630, 640 of the plurality of representative MCL fitting curves 310, 410, 610, 620, 630, 640 as a selected most comfortable level (MCL) fitting curve based on the user adjustment feedback 500, 718, 720, 722, 900, 1000. The method 500, 700, 800, 900, 1000 comprises uploading 728, by the at least one processor, the selected MCL fitting curve 620, 630, 640 to a hearing aid 130 communicatively coupled to the computing device 110.

In certain embodiments, the automatically selecting 714 the one 610 of the plurality of representative MCL fitting curves 310, 410, 610, 620, 630, 640 as an initial default MCL fitting curve 610 is based on a user age. In an exemplary embodiment, the method 500, 700, 800, 900, 1000 further comprising applying, by the hearing aid 130, the selected MCL fitting curve 620, 630, 640 to generate an acoustic output. In a representative embodiment, the smoothing process comprises averaging all MCL values in the subset of existing MCL curves corresponding with one user age for each of a predetermined plurality of frequencies, to derive the plurality of representative MCL fitting curves 310, 410, 610, 620, 630, 640. In various embodiments, the smoothing process comprises averaging MCL values in the subset of existing MCL curves corresponding with one user age for each of a predetermined plurality of frequencies except for one of the predetermined plurality of frequencies having a highest variation of the MCL values, to derive the plurality of representative MCL fitting curves 310, 410, 610, 620, 630, 640. In certain embodiments, the smoothing process comprises applying principal component analysis to reduce a dimensionality of the subset of existing MCL curves from a first number of frequencies to a second number of frequencies that is less than the first number of frequencies, and uncompressing the second number of frequencies back to the first number of frequencies with some loss of information to derive the plurality of representative MCL fitting curves 310, 410, 610, 620, 630, 640. In an exemplary embodiment, the smoothing process comprises applying an encoder-decoder neural network architecture to the subset of existing MCL curves to derive the plurality of representative MCL fitting curves 310, 410, 610, 620, 630, 640.

In a representative embodiment, the user adjustment feedback 500, 718, 720, 722, 900, 1000 is received in response to a loudness balance test 500, 720, the loudness balance comprising: (1) separately outputting 508, 512 a low frequency sound and a high frequency sound from the hearing aid 130; (2) receiving a user input 516 indicating a user of the hearing aid 130 perceived one the low frequency sound and the high frequency sound as louder; (3) increasing a level 518, 522 of the one of the low frequency sound or the high frequency sound that was perceived as louder; and (4) repeating (1)-(3) until a defined condition is met 528. In various embodiments, the user adjustment feedback 500, 718, 720, 722, 900, 1000 is results of an otoacoustic emissions (OAE) test 718, 900. In certain embodiments, the user adjustment feedback 500, 718, 720, 722, 900, 1000 corresponds with received manual adjustments 722, 1000 of one or more user interface elements 650-675 of a plurality of user interface elements 650-675, each of the plurality of user interface elements 650-675 corresponding with a respective frequency range and defining a level of the respective frequency range.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing consumers with tools to perform hearing aid fittings.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A system comprising:
a hearing database configured to store hearing data comprising a set of existing most comfortable level (MCL) curves; and
a computing device comprising at least one processor, the computing device communicatively coupled to the hearing database and a hearing aid, the at least one processor configured to:
extract a subset of existing most comfortable level (MCL) curves from the set of existing MCL curves, wherein the subset of existing MCL curves is less than the set of MCL curves;
execute a smoothing process on the subset of existing MCL curves to derive a plurality of representative most comfortable level (MCL) fitting curves;
automatically select a first one of the plurality of representative MCL fitting curves as an initial default most comfortable level (MCL) fitting curve;
receive user adjustment feedback to the initial default MCL fitting curve;
automatically select a second one of the plurality of representative MCL fitting curves as a selected most comfortable level (MCL) fitting curve based on the user adjustment feedback; and
upload the selected MCL fitting curve to the hearing aid.

2. The system of claim 1, wherein the one of the plurality of representative MCL fitting curves is automatically selected as the initial default MCL fitting curve based on a user age.

3. The system of claim 1, further comprising the hearing aid configured to apply the selected MCL fitting curve to generate an acoustic output.

4. The system of claim 1, wherein the at least one processor configured to execute the smoothing process to average all MCL values in the subset of existing MCL curves corresponding with one user age for each of a predetermined plurality of frequencies, to derive the plurality of representative MCL fitting curves.

5. The system of claim 1, wherein the at least one processor configured to execute the smoothing process to average MCL values in the subset of existing MCL curves corresponding with one user age for each of a predetermined plurality of frequencies except for one of the predetermined plurality of frequencies having a highest variation of the MCL values, to derive the plurality of representative MCL fitting curves.

6. The system of claim 1, wherein the at least one processor configured to execute the smoothing process to:
apply principal component analysis to reduce a dimensionality of the subset of existing MCL curves from a first number of frequencies to a second number of frequencies that is less than the first number of frequencies, and
uncompress the second number of frequencies back to the first number of frequencies with some loss of information to derive the plurality of representative MCL fitting curves.

7. The system of claim 1, wherein the at least one processor configured to execute the smoothing process to apply an encoder-decoder neural network architecture to the subset of existing MCL curves to derive the plurality of representative MCL fitting curves.

8. The system of claim 1, wherein the user adjustment feedback is received in response to a loudness balance test, the loudness balance comprising:
(1) separately outputting a low frequency sound and a high frequency sound from the hearing aid;
(2) receiving a user input indicating a user of the hearing aid perceived one the low frequency sound and the high frequency sound as louder;
(3) increasing a level of the one of the low frequency sound or the high frequency sound that was perceived as louder; and
(4) repeating (1)-(3) until a defined condition is met.

9. The system of claim 1, wherein the user adjustment feedback is results of an otoacoustic emissions (OAE) test.

10. The system of claim 1, wherein the user adjustment feedback corresponds with received manual adjustments of one or more user interface elements of a plurality of user interface elements, each of the plurality of user interface elements corresponding with a respective frequency range and defining a level of the respective frequency range.

11. A method comprising:
extracting, by at least one processor of a computing device, a subset of existing most comfortable level (MCL) curves from a set of existing most comfortable level (MCL) curves stored at a hearing database communicatively coupled to the computing device, wherein the subset of existing MCL curves is less than the set of MCL curves;
deriving, by the at least one processor executing a smoothing process on the subset of existing MCL curves, a plurality of representative most comfortable level (MCL) fitting curves;
automatically selecting, by the at least one processor, a first one of the plurality of representative MCL fitting curves as an initial default most comfortable level (MCL) fitting curve;
receiving, by the at least one processor, user adjustment feedback to the initial default MCL fitting curve;
automatically selecting, by the at least one processor, a second one of the plurality of representative MCL fitting curves as a selected most comfortable level (MCL) fitting curve based on the user adjustment feedback; and
uploading, by the at least one processor, the selected MCL fitting curve to a hearing aid communicatively coupled to the computing device.

12. The method of claim 11, wherein the automatically selecting the one of the plurality of representative MCL fitting curves as an initial default MCL fitting curve is based on a user age.

13. The method of claim 11, further comprising applying, by the hearing aid, the selected MCL fitting curve to generate an acoustic output.

14. The method of claim 11, wherein the smoothing process comprises averaging all MCL values in the subset of existing MCL curves corresponding with one user age for each of a predetermined plurality of frequencies, to derive the plurality of representative MCL fitting curves.

15. The method of claim 11, wherein the smoothing process comprises averaging MCL values in the subset of existing MCL curves corresponding with one user age for each of a predetermined plurality of frequencies except for one of the predetermined plurality of frequencies having a highest variation of the MCL values, to derive the plurality of representative MCL fitting curves.

16. The method of claim 11, wherein the smoothing process comprises:
applying principal component analysis to reduce a dimensionality of the subset of existing MCL curves from a first number of frequencies to a second number of frequencies that is less than the first number of frequencies, and
uncompressing the second number of frequencies back to the first number of frequencies with some loss of information to derive the plurality of representative MCL fitting curves.

17. The method of claim 11, wherein the smoothing process comprises applying an encoder-decoder neural network architecture to the subset of existing MCL curves to derive the plurality of representative MCL fitting curves.

18. The method of claim 11, wherein the user adjustment feedback is received in response to a loudness balance test, the loudness balance comprising:
(1) separately outputting a low frequency sound and a high frequency sound from the hearing aid;
(2) receiving a user input indicating a user of the hearing aid perceived one the low frequency sound and the high frequency sound as louder;
(3) increasing a level of the one of the low frequency sound or the high frequency sound that was perceived as louder; and
(4) repeating (1)-(3) until a defined condition is met.

19. The method of claim 11, wherein the user adjustment feedback is results of an otoacoustic emissions (OAE) test.

20. The method of claim 11, wherein the user adjustment feedback corresponds with received manual adjustments of one or more user interface elements of a plurality of user interface elements, each of the plurality of user interface elements corresponding with a respective frequency range and defining a level of the respective frequency range.
